(19) United States
(12) Patent Application Publication
(10) Pub. No.: US 2023/0398103 A1
(43) Pub. Date: Dec. 14, 2023

(54) ANTIBIOTIC ADJUVANTS FOR GRAM NEGATIVE BACTERIA

(71) Applicants: Laura Miller Conrad, Redwood City, CA (US); Minh Tran, San Francisco, CA (US)

(72) Inventors: Laura Miller Conrad, Redwood City, CA (US); Minh Tran, San Francisco, CA (US)

(21) Appl. No.: 18/330,912

(22) Filed: Jun. 7, 2023

Related U.S. Application Data
(60) Provisional application No. 63/351,737, filed on Jun. 13, 2022.

(51) Int. Cl. A61K 31/44; A61K 38/12; A61K 31/444; A61P 31/04

(52) U.S. Cl. CPC A61K 31/44; A61K 38/12; A61K 31/444; A61P 31/04

(57) ABSTRACT

Various aspects disclosed relate to an antibiotic adjuvant. The adjuvant includes the structure according to Formula I:

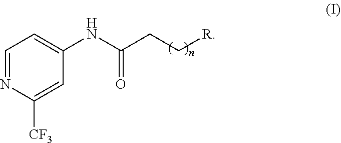

In Formula I, R can be selected from:
a substituted or unsubstituted (C1-C13)hydrocarbyl;
the structure according to Formula II

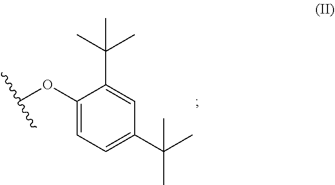 [SMILES: *Oc1ccc(C(C)(C)C)cc1C(C)(C)C]

the structure according to Formula III

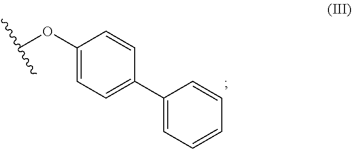 [SMILES: *Oc1ccc(-c2ccccc2)cc1]

the structure according to Formula IV

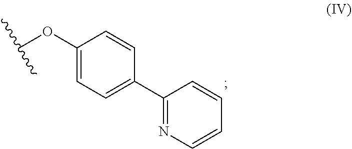 [SMILES: *Oc1ccc(-c2ccccn2)cc1]

and
n is in a range of 1-10.

### CROSS-REFERENCE TO RELATED APPLICATION

This application claims the benefit of priority to U.S. Provisional Patent Application Ser. No. 63/351,737 entitled “ANTIBIOTIC ADJUVANTS FOR GRAM NEGATIVE BACTERIA.” filed Jun. 13, 2022, the disclosure of which is incorporated herein in its entirety by reference.

### STATEMENT OF GOVERNMENT SUPPORT

This invention was made with Government support under GM118199 awarded by National Institute of Health. The U.S. Government has certain rights in this invention.

### BACKGROUND

Multidrug-resistant organisms are bacteria that have become resistant to certain antibiotics, and these antibiotics can no longer be used to control or kill the bacteria. Antibiotics are important medicines. They help fight infections that are caused by bacteria. Bacteria that resist treatment with more than one antibiotic are called multidrug-resistant organisms (MDROs). Multidrug-resistant organisms are found mainly in hospitals and long-term care facilities. They often affect people who are older or very ill and can cause severe infections.

### SUMMARY OF THE DISCLOSURE

Various aspects disclosed relate to an antibiotic adjuvant. The adjuvant includes the structure according to Formula I:

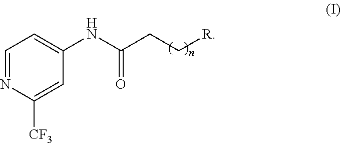

In Formula I, R can be selected from:
a substituted or unsubstituted (C1-C13)hydrocarbyl;
the structure according to Formula II

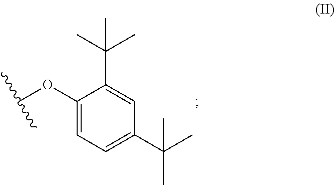 [SMILES: *Oc1ccc(C(C)(C)C)cc1C(C)(C)C]

the structure according to Formula III

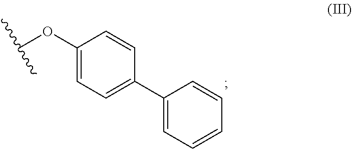 [SMILES: *Oc1ccc(-c2ccccc2)cc1]

the structure according to Formula IV

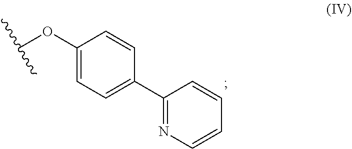 [SMILES: *Oc1ccc(-c2ccccn2)cc1]

and
n is in a range of 1-10.

### DETAILED DESCRIPTION OF THE DISCLOSURE

Reference will now be made in detail to certain aspects of the disclosed subject matter. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference. In the event of inconsistent usages between this document and those documents so incorporated by reference, the usage in the incorporated reference should be considered supplementary to that of this document; for irreconcilable inconsistencies, the usage in this document controls.

In the methods described herein, the acts can be carried out in any order without departing from the principles of the disclosure, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

The term “about” as used herein can allow for a degree of variability in a value or range, for example, within 10%, within 5%, or within 1% of a stated value or of a stated limit of a range, and includes the exact stated value or range. The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The term “substantially free of” as used herein can mean having none or having a trivial amount of, such that the amount of material present does not affect the material properties of the composition including the material, such that about 0 wt % to about 5 wt % of the composition is the material, or about 0 wt % to about 1 wt %, or about 5 wt % or less, or less than or equal to about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

The term “organic group” as used herein refers to any carbon-containing functional group. Examples can include an oxygen-containing group such as an alkoxy group, aryloxy group, aralkyloxy group, oxo(carbonyl) group; a carboxyl group including a carboxylic acid, carboxylate, and a carboxylate ester; a sulfur-containing group such as an alkyl and aryl sulfide group; and other heteroatom-containing groups. Non-limiting examples of organic groups include OR, OOR, OC(O)N(R)₂, CN, CF₃, OCF₃, R, C(O), methylenedioxy, ethylenedioxy. N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, C(═NOR)R, and substituted or unsubstituted (C₁-C₁₀₀)hydrocarbyl, wherein R can be hydrogen (in examples that include other carbon atoms) or a carbon-based moiety, and wherein the carbon-based moiety can be substituted or unsubstituted.

The term “substituted” as used herein in conjunction with a molecule or an organic group as defined herein refers to the state in which one or more hydrogen atoms contained therein are replaced by one or more non-hydrogen atoms. The term “functional group” or “substituent” as used herein refers to a group that can be or is substituted onto a molecule or onto an organic group. Examples of substituents or functional groups include, but are not limited to, a halogen (e.g., F, Cl, Br, and I); an oxygen atom in groups such as hydroxy groups, alkoxy groups, aryloxy groups, aralkyloxy groups, oxo(carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, hydroxyamines, nitriles, nitro groups, N-oxides, hydrazides, azides, and enamines; and other heteroatoms in various other groups. Non-limiting examples of substituents that can be bonded to a substituted carbon (or other) atom include F, Cl, Br, I, OR, OC(O)N(R)₂, CN, NO, NO₂, ONO₂, azido, CF₃, OCF₃, R, O (oxo), S (thiono), C(O), S(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, and C(═NOR)R, wherein R can be hydrogen or a carbon-based moiety; for example, R can be hydrogen, (C₁-C₁₀₀)hydrocarbyl, alkyl, acyl, cycloalkyl, aryl, aralkyl, heterocyclyl, heteroaryl, or heteroarylalkyl; or wherein two R groups bonded to a nitrogen atom or to adjacent nitrogen atoms can together with the nitrogen atom or atoms form a heterocyclyl.

The term “alkyl” as used herein refers to straight chain and branched alkyl groups and cycloalkyl groups having from 1 to 40 carbon atoms, 1 to about 20 carbon atoms, 1 to 12 carbons or, in some respects, from 1 to 8 carbon atoms. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. As used herein, the term “alkyl” encompasses n-alkyl, isoalkyl, and anteisoalkyl groups as well as other branched chain forms of alkyl. Representative substituted alkyl groups can be substituted one or more times with any of the groups listed herein, for example, amino, hydroxy, cyano, carboxy, nitro, thio, alkoxy, and halogen groups.

The term “alkenyl” as used herein refers to straight and branched chain and cyclic alkyl groups as defined herein, except that at least one double bond exists between two carbon atoms. Thus, alkenyl groups have from 2 to 40 carbon atoms, or 2 to about 20 carbon atoms, or 2 to 12 carbon atoms or, in some respects, from 2 to 8 carbon atoms. Examples include, but are not limited to vinyl, —CH═CH(CH₃), —CH═C(CH₃)₂, —C(CH₃)═CH₂, —C(CH₃)═CH(CH₃), —C(CH₂CH₃)═CH₂, cyclohexenyl, cyclopentenyl, cyclohexadienyl, butadienyl, pentadienyl, and hexadienyl among others.

The term “alkynyl” as used herein refers to straight and branched chain alkyl groups, except that at least one triple bond exists between two carbon atoms. Thus, alkynyl groups have from 2 to 40 carbon atoms, 2 to about 20 carbon atoms, or from 2 to 12 carbons or, in some respects, from 2 to 8 carbon atoms. Examples include, but are not limited to —C≡CH, —C≡C(CH₃), —C≡C(CH₂CH₃), —CH₂C≡CH, —CH₂C≡C(CH₃), and —CH₂C≡C(CH₂CH₃) among others.

The term “acyl” as used herein refers to a group containing a carbonyl moiety wherein the group is bonded via the carbonyl carbon atom. The carbonyl carbon atom is bonded to a hydrogen forming a “formyl” group or is bonded to another carbon atom, which can be part of an alkyl, aryl, aralkyl cycloalkyl, cycloalkylalkyl, heterocyclyl, heterocyclylalkyl, heteroaryl, heteroarylalkyl group or the like. An acyl group can include 0 to about 12, 0 to about 20, or 0 to about 40 additional carbon atoms bonded to the carbonyl group. An acyl group can include double or triple bonds within the meaning herein. An acryloyl group is an example of an acyl group. An acyl group can also include heteroatoms within the meaning herein. A nicotinoyl group (pyridyl-3-carbonyl) is an example of an acyl group within the meaning herein. Other examples include acetyl, benzoyl, phenylacetyl, pyridylacetyl, cinnamoyl, and acryloyl groups and the like. When the group containing the carbon atom that is bonded to the carbonyl carbon atom contains a halogen, the group is termed a “haloacyl” group. An example is a trifluoroacetyl group.

The term “cycloalkyl” as used herein refers to cyclic alkyl groups such as, but not limited to, cyclopropyl, cyclobutyl, cyclopentyl, cyclohexyl, cycloheptyl, and cyclooctyl groups. In some respects, the cycloalkyl group can have 3 to about 8-12 ring members, whereas in other aspects the number of ring carbon atoms range from 3 to 4, 5, 6, or 7. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. Cycloalkyl groups also include rings that are substituted with straight or branched chain alkyl groups as defined herein. Representative substituted cycloalkyl groups can be mono-substituted or substituted more than once, such as, but not limited to, 2,2-, 2,3-, 2,4-2,5- or 2,6-disubstituted cyclohexyl groups or mono-, di- or tri-substituted norbornyl or cycloheptyl groups, which can be substituted with, for example, amino, hydroxy, cyano, carboxy, nitro, thio, alkoxy, and halogen groups. The term “cycloalkenyl” alone or in combination denotes a cyclic alkenyl group.

The term “aryl” as used herein refers to cyclic aromatic hydrocarbon groups that do not contain heteroatoms in the ring. Thus, aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some respects, aryl groups contain about 6 to about 14 carbons in the ring portions of the groups. Aryl groups can be unsubstituted or substituted, as defined herein. Representative substituted aryl groups can be mono-substituted or substituted more than once, such as, but not limited to, a phenyl group substituted at any one or more of 2-, 3-, 4-, 5-, or 6-positions of the phenyl ring, or a naphthyl group substituted at any one or more of 2- to 8-positions thereof.

The term “aralkyl” as used herein refers to alkyl groups as defined herein in which a hydrogen or carbon bond of an alkyl group is replaced with a bond to an aryl group as defined herein. Representative aralkyl groups include benzyl and phenylethyl groups and fused (cycloalkylaryl)alkyl groups such as 4-ethyl-indanyl. Aralkenyl groups are alkenyl groups as defined herein in which a hydrogen or carbon bond of an alkyl group is replaced with a bond to an aryl group as defined herein.

The term “alkoxy” as used herein refers to an oxygen atom connected to an alkyl group, including a cycloalkyl group, as are defined herein. Examples of linear alkoxy groups include but are not limited to methoxy, ethoxy, propoxy, butoxy, pentyloxy, hexyloxy, and the like. Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like. An alkoxy group can include about 1 to about 12, about 1 to about 20, or about 1 to about 40 carbon atoms bonded to the oxygen atom, and can further include double or triple bonds, and can also include heteroatoms. For example, an allyloxy group or a methoxyethoxy group is also an alkoxy group within the meaning herein, as is a methylenedioxy group in a context where two adjacent atoms of a structure are substituted therewith.

The term “amine” as used herein refers to primary, secondary, and tertiary amines having, e.g., the formula N(group)₃ wherein each group can independently be H or non-H, such as alkyl, aryl, and the like. Amines include but are not limited to R—NH₂, for example, alkylamines, arylamines, alkylarylamines; R₂NH wherein each R is independently selected, such as dialkylamines, diarylamines, aralkylamines, heterocyclylamines and the like; and R₃N wherein each R is independently selected, such as trialkylamines, dialkylarylamines, alkyldiarylamines, triarylamines, and the like. The term “amine” also includes ammonium ions as used herein.

The terms “halo.” “halogen,” or “halide” group, as used herein, by themselves or as part of another substituent, mean, unless otherwise stated, a fluorine, chlorine, bromine, or iodine atom.

The term “haloalkyl” group, as used herein, includes mono-halo alkyl groups, poly-halo alkyl groups wherein all halo atoms can be the same or different, and per-halo alkyl groups, wherein all hydrogen atoms are replaced by halogen atoms, such as fluoro. Examples of haloalkyl include trifluoromethyl, 1,1-dichloroethyl, 1,2-dichloroethyl, 1,3-dibromo-3.3-difluoropropyl, perfluorobutyl, and the like.

The term “hydrocarbon” or “hydrocarbyl” as used herein refers to a molecule or functional group that includes carbon and hydrogen atoms. The term can also refer to a molecule or functional group that normally includes both carbon and hydrogen atoms but wherein all the hydrogen atoms are substituted with other functional groups. The term “hydrocarbyl” refers to a functional group derived from a straight chain, branched, or cyclic hydrocarbon, and can be alkyl, alkenyl, alkynyl, aryl, cycloalkyl, acyl, or any combination thereof. Hydrocarbyl groups can be shown as (Cₐ-C_{b})hydrocarbyl, wherein a and b are integers and mean having any of a to b number of carbon atoms. For example, (C₁-C₄)hydrocarbyl means the hydrocarbyl group can be methyl (C₁), ethyl (C₂), propyl (C₃), or butyl (C₄), and (C₀-C_{b})hydrocarbyl means in certain aspects there is no hydrocarbyl group. A hydrocarbylene group is a diradical hydrocarbon, e.g., a hydrocarbon that is bonded at two locations.

According to various aspects of the instant disclosure an adjuvant is described that can be used to enhance the performance of an antibiotic. In particular, the adjuvants described herein can be particularly effective in enhancing the effectiveness of cationic antimicrobial peptides. Examples of cationic antimicrobial peptides include colistin and polymyxin B. These cationic antimicrobial peptides are typically used as “last resort” antibiotics for multidrug resistant bacteria such as Pseudomonas aeruginosa. Other bacteria that the adjuvant can be used in conjunction with can include gram negative bacteria such as Acinetobacter baumanii, Klebsiella pneumoniae, Burkholderia cepacia, Chronobacterium violaceum, Vibrio harveyi, Neisseria gonorrhoeae, Neisseria meningitidis, Bordetella pertussis, Haemophilus influenzae, Legionella pneumophila, Brucella sp., Francisella sp., Agrobacterium sp., Escherichia coli, Salmonella sp., Shigella spp., Proteus spp., or Yersinia pestis.

According to the instant disclosure, an antibiotic adjuvant can include the structure according to Formula I:

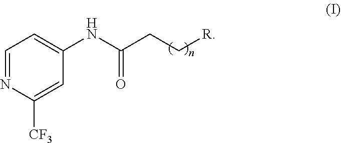

In Formula I, R can be a substituted or unsubstituted (C1-C13)hydrocarbyl. The substituted or unsubstituted (C1-C13)hydrocarbyl can be a (C1-C13)alkyl. The substituted or unsubstituted (C1-C13)alkyl can be a (C1-C13)haloalkyl. The value n can be in a range of 1 to 10. The (C1-C13)haloalkyl can be a —C₃H₆Br. In other examples, R can be represented by the structure according to Formula II

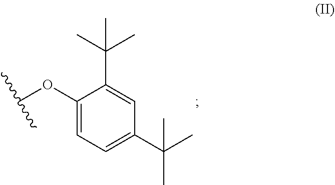 [SMILES: *Oc1ccc(C(C)(C)C)cc1C(C)(C)C]

the structure according to Formula III

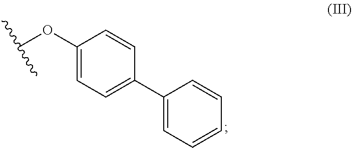 [SMILES: *Oc1ccc(-c2ccccc2)cc1]

and/or the structure according to Formula IV

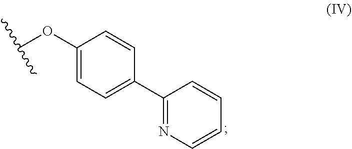 [SMILES: *Oc1ccc(-c2ccccn2)cc1]

The adjuvant of the instant disclosure can be delivered as part of a pharmaceutical composition. The pharmaceutical composition includes the antibiotic adjuvant and the antibiotic such as the aforementioned antibiotic. Notably in some aspects owing to the adjuvant, less antibiotic may need to be administered to have a beneficial effect than would need to added without the adjuvant. While not intending to be limited to any theory, it is thought that the composition functions by inhibiting ArnA in the cationic antibiotic peptide resistance pathway.

The composition can be used in conjunction with methods of treatment, inhibition and prophylaxis by administration to a subject of an effective amount of the compound or a pharmaceutical composition of the disclosure. In some aspects, the compound is substantially purified (e.g., substantially free from substances that limit its effect or produce undesired side-effects). The subject can be an animal, including but not limited to mammals, amphibians, birds, and fish. The subject is more preferably either a mammal, including but not limited to animals such as cows, pigs, horses, cats, dogs, etc., or an avian species, including but not limited to chickens, ducks and other domestic poultry. In some aspects the subject is most preferably a human.

Formulations and methods of administration that can be employed with the compound or compound compositions, additional appropriate formulations and routes of administration can be selected from among those described herein below. The compound or compound compositions of the disclosure may be administered therapeutically, such as, for example, in the case of infection of a susceptible patient with burn or other traumatic wound injury or lung infection, such as in a cystic fibrosis patient infected with P. aeruginosa or B. cepacia separately or in combination. Alternatively, the compound or compound compositions may be administered prophylactically, such as, for example, to prevent opportunistic gram negative bacterial infection, such as by P. aeruginosa, prior to surgery, dental work, or implantation of a medical device such as a catheter or ventilator tube continuously, such as, for example in the case of an immunosuppressed or immunocompromised patient.

Various delivery systems are known and can be used to administer the disclosed compounds, e.g., encapsulation in liposomes, microparticles, microcapsules. Methods of introduction include, but are not limited to, topical, intradermal, intramuscular, intraperitoneal, intravenous, subcutaneous, inhalation, intranasal, epidural, and oral routes. The compositions may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compositions of the disclosure into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

In an example, it may be desirable to administer the pharmaceutical compositions of the disclosure locally to the area in need of treatment; this may be achieved by, for example, and not by way of limitation, local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers.

In another aspect, the composition can be delivered in a vesicle, in particular a liposome. In yet another aspect, the composition can be delivered in a controlled release system. In one aspect, a pump may be used. In another aspect, polymeric materials can be used In yet another aspect, a controlled release system can be placed in proximity of the therapeutic target, i.e., the brain, thus requiring only a fraction of the systemic dose.

The present disclosure also provides pharmaceutical compositions. Such compositions comprise a therapeutically effective amount of compound and a pharmaceutically acceptable carrier. In a specific aspect, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the compound together with a suitable amount of carrier so as to provide the form for proper administration to the patient. The formulation should suit the mode of administration.

In an example, the composition is formulated in accordance with routine procedures as a pharmaceutical composition adapted for intravenous, topical or pulmonary administration to human beings. Typically, compositions for such administration are solutions in sterile isotonic aqueous buffer. Where necessary, the composition may also include a solubilizing agent and a local anesthetic such as lignocanme to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compositions of the disclosure can be formulated as neutral or salt forms, Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

The amount of the composition of the disclosure that will be effective in the treatment, inhibition and prevention of a disease or disorder can be determined by standard clinical techniques. Additionally, in vitro assays may optionally be employed to help identify optimal dosage ranges. The precise dose to be employed in the formulation will also depend on the route of administration, and the seriousness of the disease or disorder, and should be decided according to the judgment of the practitioner and each patient's circumstances. Effective doses may be extrapolated from dose-response curves derived from in vitro or animal model test systems.

As described herein above, the compound or compound compositions of the disclosure may be administered alone or in combination with other compounds, such as adjuvants. In one aspect the compounds may be administered in combination with one or more antibiotics, for example, gentamicin, tobramycin, colistin, and fluoroquinolins. Combinations may be administered either concomitantly, e.g., as an admixture, separately but simultaneously or concurrently; or sequentially. This includes presentations in which the combined agents are administered together as a therapeutic mixture, and also procedures in which the combined agents are administered separately but simultaneously, e.g., as through separate intravenous lines into the same individual. Administration “in combination” further includes the separate administration of one of the compounds or agents given first, followed by the second.

Compounds of the disclosure can be used in industrial settings to inhibit biofilm production and/or to remove antibiotic resistant bacteria, such as in a hospital or other public setting. For example, the compounds of the disclosure can be used to remove biofilms that have grown in moist and warm environments, such as showers, water and sewage pipes, cooling or heating water systems, (e.g., cooling towers), marine engineering systems, such as, for example, pipelines of the offshore oil and gas industry.

### Examples

Various aspects of the present disclosure can be better understood by reference to the following Examples which are offered by way of illustration. The present disclosure is not limited to the Examples given herein.

Methods: General chemical methods. Unless otherwise stated, reactions were performed in flame-dried glassware fitted with rubber septa under nitrogen atmosphere and were stirred with Teflon™ coated magnetic stirring bars. Liquid reagents and solvents were transferred via syringe using standard Schlenk techniques. Reaction solvents were dried by passage over a column of activated alumina if noted. All other solvents and reagents were used as received unless otherwise noted. Reaction temperatures above 23° C. refer to oil bath temperature, which was controlled by an OptiCHEM or IKA RCT Basic temperature modulator. Thin layer chromatography was performed using SiliCycle silica gel 60 F-254 precoated plates (0.25 mm) and visualized by UV irradiation and anisaldehyde or potassium permanganate stain. Sorbent standard silica gel (particle size 40-63 μm) or SiliCycle Silica-P silica gel (particle size 40-63 μm) was used for flash chromatography.

### General Procedure A

The respective 4-aminopyridine (1 equiv). CH₂Cl₂ (0.15 M), and pyridine (2 equiv) were combined in a flame-dried flask. The reaction mixture was cooled to 0° C., and the acid chloride (1 equiv) was added dropwise. The reaction mixture was allowed to warm to room temperature over 3-5 h. The reaction was then quenched with saturated aqueous NaHCO₃. The layers were separated, and the aqueous layer was extracted 3× with CH₂Cl₂. The combined organic layer was washed sequentially with 1 M HCl and brine, then dried over MgSO₄ and concentrated. The crude product was purified by column chromatography.

### General Procedure B

The alkyl halide (1 equiv), anhydrous potassium iodide (12 equiv), anhydrous potassium carbonate (7.5 equiv), and the aryl nucleophile (3.8 equiv) were dissolved in DMF (0.68 M). The reaction was stirred for 3-5 days at room temperature until complete by TLC. The reaction was quenched with water and extracted with CH₂Cl₂. The combined organic layer was washed sequentially with saturated aqueous NaHCO₃ (2×), 1 M HCl and brine. The solution was dried over MgSO₄ and concentrated. The crude product was purified by column chromatography.

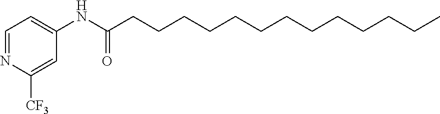 [SMILES: CCCCCCCCCCCCCC(=O)Nc1ccnc(C(F)(F)F)c1]

N-(2-(trifluoromethyl)pyridin-4-yl)tetradecanamide (S1). Prepared from 4-amino-2-(trifluoromethyl)pyridine and tetradecanoyl chloride using general procedure A to furnish S1 in a 56% yield.

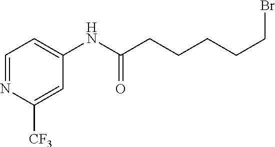 [SMILES: O=C(CCCCCBr)Nc1ccnc(C(F)(F)F)c1]

6-Bromo-N-(2-(trifluoromethyl)pyridin-4-yl)-hexanamide (S2). Prepared from 4-amino-2-(trifluoromethyl)pyridine and 6-bromohexanoyl chloride using general procedure A to furnish S2 in a 90% yield.

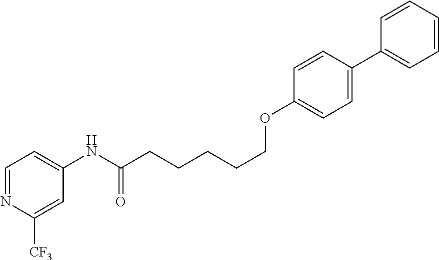 [SMILES: O=C(CCCCCOc1ccc(-c2ccccc2)cc1)Nc1ccnc(C(F)(F)F)c1]

6-([1,1′-biphenyl]-4-yloxy)-N-(2-(trifluoromethyl)pyridin-4-yl)hexanamide (S3). Prepared from amide S2 and 4-phenylphenol using general procedure B to furnish S3.

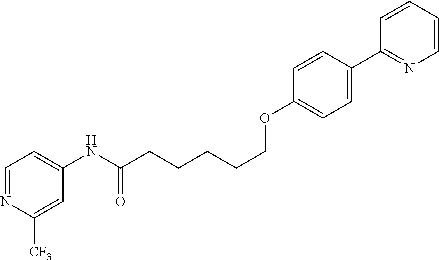 [SMILES: O=C(CCCCCOc1ccc(-c2ccccn2)cc1)Nc1ccnc(C(F)(F)F)c1]

6-(4-(pyridin-2-yl)phenoxy)-N-(2-(trifluoromethyl)pyridin-4-yl)hexanamide (S4). Prepared from amide S2 and 4-(pyridin-2-yl)phenol using general procedure B to furnish S4 in a 30% yield.

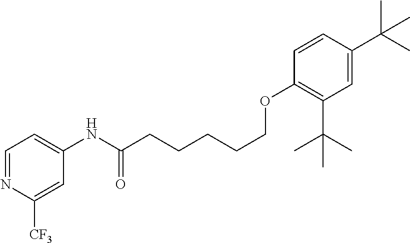 [SMILES: CC(C)(C)c1ccc(OCCCCCC(=O)Nc2ccnc(C(F)(F)F)c2)c(C(C)(C)C)c1]

6-(2,4-di-tert-butylphenoxy)-N-(2-(trifluoromethyl)pyridin-4-yl)hexanamide (S5). Prepared from amide S2 and 2,4-ditertbutylphenol using general procedure B to furnish S5 in a 32% yield.

### IC50 Testing

Overnight cultures of P. aeruginosa PA14 were back diluted into fresh LB to an OD600 of 2.5×10⁻³. Using polypropylene 96 well plates (Corning 3879), the potential antibiotic adjuvant was assayed with a 3-fold dilution starting with a 250 μM concentration. The concentration of colistin was held constant at sublethal concentration (0.25 to 0.375 μg/mL), using a freshly prepared colistin stock and minimizing transfers of colistin-containing solutions. After 17 h of aerobic growth at 37° C. 300 rpm, the absorbance at 600 nm was recorded.

The terms and expressions that have been employed are used as terms of description and not of limitation, and there is no intention in the use of such terms and expressions of excluding any equivalents of the features shown and described or portions thereof, but it is recognized that various modifications are possible within the scope of the aspects of the present disclosure. Thus, it should be understood that although the present disclosure has been specifically disclosed by specific aspects and optional features, modification and variation of the concepts herein disclosed may be resorted to by those of ordinary skill in the art, and that such modifications and variations are considered to be within the scope of aspects of the present disclosure.

### Exemplary Aspects.

The following exemplary aspects are provided, the numbering of which is not to be construed as designating levels of importance:

Aspect 1 provides an antibiotic adjuvant comprising the structure according to Formula I:

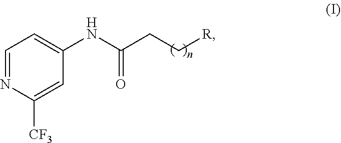

wherein R is selected from:
   a substituted or unsubstituted (C1-C13)hydrocarbyl;
   the structure according to Formula II

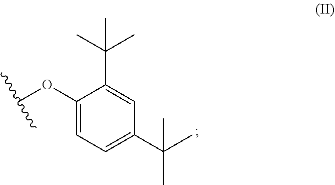 [SMILES: *Oc1ccc(C(C)(C)C)cc1C(C)(C)C]

the structure according to Formula III

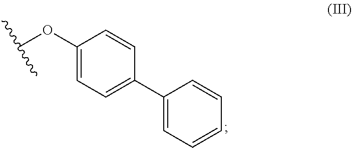 [SMILES: *Oc1ccc(-c2ccccc2)cc1]

the structure according to Formula IV

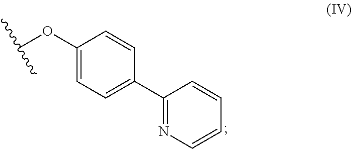 [SMILES: *Oc1ccc(-c2ccccn2)cc1]

n is in a range of 1-10.

Aspect 2 provides the antibiotic adjuvant of Aspect 1, wherein the substituted or unsubstituted (C1-C13)hydrocarbyl is a (C1-C13)alkyl.

Aspect 3 provides the antibiotic adjuvant of Aspect 2, wherein the substituted or unsubstituted (C1-C13)alkyl is a (C1-C13)haloalkyl.

Aspect 4 provides the antibiotic adjuvant of Aspect 3, wherein the (C1-C13)haloalkyl is —C3H6Br.

Aspect 5 provides a composition comprising:
the antibiotic adjuvant of any one of Aspects 1-4; and
an antibiotic.

Aspect 6 provides the composition of Aspect 5, wherein the antibiotic comprises colistin, polymyxin B, or a mixture thereof.

Aspect 7 provides a method for inhibiting, in a subject, gram negative bacteria biofilm production, virulence factor production, pyocyanin production, or quorum sensing, the method comprising:
providing a composition comprising a compound of Aspect 5 or 6; and
contacting at least one gram negative bacteria with the composition.

Aspect 8 provides the method of Aspect 7, wherein the gram negative bacteria is selected from Acinetobacter baumanii, Klebsiella pneumoniae, Burkholderia cepacia, Chromobacterium violaceum, Vibrio harveyi, Neisseria gonorrhoeae, Neisseria meningitidis, Bordetella pertussis, Haemophilus influenzae, Legionella pneumophila, Brucella sp., Francisella sp., Agrobacterium sp., Escherichia coli, Salmonella sp., Shigella spp., Proteus spp., or Yersinia pestis.

Aspect 9 provides the method of Aspect 8, wherein the Pseudomonas is P. aeruginosa.

Aspect 10 provides the method of any one of Aspects 7-9, wherein the composition inhibits ArnA in a cationic antibiotic peptide resistance pathway.

Aspect 11 provides the method of any one of Aspects 7-10, wherein the subject is selected from an animal, a human, or both.

Aspect 12 provides the method of any one of Aspects 7-11, wherein the subject has an infection selected from a respiratory illness, pulmonary tract infection, a urinary tract infection, a catheter-associated infection, a blood infection, a middle ear infection, dental plaque, gingivitis, chronic sinusitis, endocarditis, eye infections, eye infections resulting from contact lens use, implanted device infection, a medical device infection, a central nervous infection, a gastrointestinal tract infection, a bone infection, a joint infection, an ear infection, an eye infection, a burn, a wound, an antibiotic resistant infection, is immunocompromised, is immunosuppressed, or has an opportunistic infection.

Aspect 13 provides the method of Aspect 12, wherein the medical device infection occurs in a patient having a catheter, a stent, a joint prosthesis, a prosthetic cardiac valve, on a ventilator or an intrauterine device.

Aspect 14 provides the method of Aspect 12, wherein the pulmonary tract infection is pneumonia.

Aspect 15 provides the method of Aspect 12, wherein the respiratory illness is Cystic Fibrosis.

Aspect 16 provides the method of any one of Aspects 14 or 15, wherein the subject has both Cystic Fibrosis and pneumonia.

Aspect 17 provides the method of any one of Aspect 5, wherein the composition is administered therapeutically, prophylactically, when a subject is undergoing surgery, implantation of medical devices, dental procedures, or a combination thereof.

Aspect 18 provides the method of any one of Aspects 7-17, wherein the composition is administered topically.

Aspect 19 provides the method of any one of Aspects 7-18, wherein the composition is administered intravenously.

Aspect 20 provides the method of any one of Aspects 7-19, wherein the composition is administered intranasally.

Aspect 21 provides the method of any one of Aspects 7-20, wherein the composition is used in an industrial setting, medical instrument, medical device, or a combination thereof.

Aspect 22 provides the method of Aspect 21, wherein the industrial setting is a work area, a chemical unit operation, a pipe, a sewage system, a pipeline, a tubing, or a filtration device.

Aspect 23 provides the method of Aspect 22, wherein the medical device is a catheter, a joint prosthesis, a prosthetic cardiac valve, a ventilator, a stent, or an intrauterine device.

Aspect 24 provides the method of Aspect 23, wherein the chemical unit operation comprises a microfluidic platform, a microelectromechanical system, a 3D printer, or a system for coating nanoparticle surfaces.

What is claimed is:

1. An antibiotic adjuvant comprising the structure according to Formula 1: 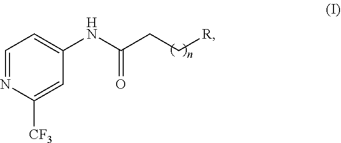
wherein R is selected from:
a substituted or unsubstituted (C1-C13)hydrocarbyl;
the structure according to Formula II 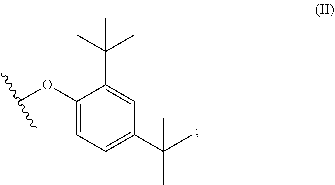 [SMILES: *Oc1ccc(C(C)(C)C)cc1C(C)(C)C]
the structure according to Formula III 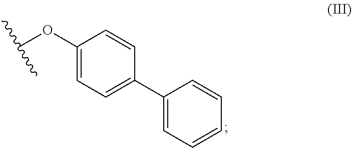 [SMILES: *Oc1ccc(-c2ccccc2)cc1]
the structure according to Formula IV 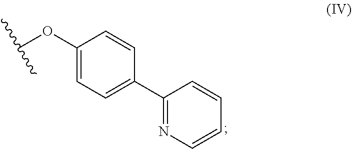 [SMILES: *Oc1ccc(-c2ccccn2)cc1]
and
n is in a range of 1-10.

2. The antibiotic adjuvant of claim 1, wherein the substituted or unsubstituted (C1-C13)hydrocarbyl is a (C1-C13)alkyl.

3. The antibiotic adjuvant of claim 2, wherein the substituted or unsubstituted (C1-C13)alkyl is a (C1-C13)haloalkyl.

4. The antibiotic adjuvant of claim 3, wherein the (C1-C13)haloalkyl is —C₃H₆Br.

5. A composition comprising:
the antibiotic adjuvant of claim 1; and
an antibiotic.

6. The composition of claim 5, wherein the antibiotic comprises colistin, polymyxin B, or a mixture thereof.

7. A method for inhibiting, in a subject, gram negative bacteria biofilm production, virulence factor production, pyocyanin production, or quorum sensing, the method comprising:
providing a composition comprising a compound of claim 5; and
contacting at least one gram negative bacteria with the composition.

8. The method of claim 7, wherein the gram negative bacteria is selected from Acinetobacter baumanii, Klebsiella pneunoniae, Burkholderia cepacia, Chromobacterium violaceum, Vibrio harveyi, Neisseria gonorrhoeae, Neisseria meningitidis, Bordetella pertussis, Haemophilus influenzae, Legionella pneumophila, Brucella sp., Francisella sp., Agrobacterium sp., Escherichia coli, Salmonella sp., Shigella spp., Proteus spp., or Yersinia pestis.

9. The method of claim 8, wherein the Pseudomonas is P. aeruginosa.

10. The method of claim 7, wherein the composition inhibits ArnA in a cationic antibiotic peptide resistance pathway.

11. The method of claim 7, wherein the subject is selected from an animal, a human, or both.

12. The method of claim 7, wherein the subject has an infection selected from a respiratory illness, pulmonary tract infection, a urinary tract infection, a catheter-associated infection, a blood infection, a middle ear infection, dental plaque, gingivitis, chronic sinusitis, endocarditis, eye infections, eye infections resulting from contact lens use, implanted device infection, a medical device infection, a central nervous infection, a gastrointestinal tract infection, a bone infection, a joint infection, an ear infection, an eye infection, a burn, a wound, an antibiotic resistant infection, is immunocompromised, is immunosuppressed, or has an opportunistic infection.

13. The method of claim 12, wherein the medical device infection occurs in a patient having a catheter, a stent, a joint prosthesis, a prosthetic cardiac valve, on a ventilator or an intrauterine device.

14. The method of claim 12, wherein the pulmonary tract infection is pneumonia.

15. The method of claim 12, wherein the respiratory illness is Cystic Fibrosis.

16. The method of claim 14, wherein the subject has both Cystic Fibrosis and pneumonia.

17. The method of claim 7, wherein the composition is administered therapeutically, prophylactically, when a subject is undergoing surgery, implantation of medical devices, dental procedures, or a combination thereof.

18. The method of claim 7, wherein the composition is administered topically.

19. The method of claim 7, wherein the composition is administered intravenously.

20. The method of claim 7, wherein the composition is administered intranasally.
